(19) United States
(12) Patent Application Publication
(10) Pub. No.: US 2016/0208107 A1
(43) Pub. Date: Jul. 21, 2016

(54) Compositions for Causing, Enhancing, and/or Expediting Oxo-Biodegradation of Articles and Methods of Production and Use Thereof

(71) Applicant: Wanda M. Weder and William F. Straeter, not Individually but solely as Trustees of The Family, Highland, IL (US)

(72) Inventor: Donald E. Weder, Highland, IL (US)

(21) Appl. No.: 15/083,802

(22) Filed: Mar. 29, 2016

Related U.S. Application Data
(63) Continuation of application No. 14/520,022, filed on Oct. 21, 2014, now abandoned.
Application No. 14/520,022 is a continuation of application No. PCT/US14/46149, filed on Jul. 10, 2014.
Application No. PCT/US14/46149 is a continuation-in-part of application No. 14/168,451, filed on Jan. 30, 2014.
Application No. 14/168,451 is a continuation-in-part of application No. 13/944,198, filed on Jul. 17, 2013.
Application No. 13/944,198 is a continuation of application No. 13/940,993, filed on Jul. 12, 2013.

(51) Int. Cl. C09D 5/00; D21H 23/22; C12N 9/02; C12N 9/08; C12N 9/16; C12N 9/14

(52) U.S. Cl. CPC C09D 5/00; C12N 9/16; C12N 9/14; C12N 9/0055; C12N 9/0065; C12N 9/0053; D21H 23/22; C12Y 110/03002; C12Y 111/01013; C12Y 111/01014; C12Y 109/03001

(57) ABSTRACT

Methods of causing, enhancing, and/or expediting oxo-biodegradation of articles using agents of oxo-biodegradation are provided.

### CROSS REFERENCE TO RELATED APPLICATIONS/INCORPORATION BY REFERENCE STATEMENT

This application is a continuation of U.S. Ser. No. 14/520,022, filed Oct. 21, 2014; which is a continuation of International Patent Application No. PCT/US14/46149, filed Jul. 10, 2014; which is a continuation-in-part of U.S. Ser. No. 14/168,451, filed Jan. 30, 2014. The International Patent Application No. PCT/US14/46149 is also a continuation-in-part of U.S. Ser. No. 13/944,198, filed Jul. 17, 2013; which is a continuation of U.S. Ser. No. 13/940,993, filed Jul. 12, 2013. The entire contents of the each of the above-referenced patent applications are hereby expressly incorporated herein by reference.

### STATEMENT REGARDING FEDERALLY SPONSORED RESEARCH AND DEVELOPMENT

Not Applicable.

### BACKGROUND

Oxo-biodegradation refers to a two-step process that combines a degradative process step with a biodegradative process step in the environment. The first step of oxo-biodegradation is an abiotic, chemical process that requires the presence of oxygen so that degradation of a material can occur through oxidation. The second step of oxo-biodegradation is a biological process that requires the presence of at least one biodegradative living organism that is capable of biodegradative consumption of the material that has been degraded in the first step. Oxo-biodegradation requires the presence of a prodegradant that speeds up the molecular breakdown of a material during the first step and that incorporates oxygen atoms into the resulting low molecular mass. This chemical change enables the further breakdown of the material in the second step by biodegradative living organism(s). In this manner, the degradation process (i.e., the first step of oxo-biodegradation) can be shortened from a period of hundreds of years to a period of just years or even months; thereafter, the rate of the biodegradative process (i.e., the second step of oxo-biodegradation) depends on the biodegradative living organism(s) present in the environment.

Currently, polymers such as polyolefins are made oxo-biodegradable through the use of oxo-biodegradable additives (also referred to in the art as prodegradants) such as metal catalysts (i.e., metal stearates like cobalt stearate, iron/ferric stearate, copper stearate, and manganese stearate) and unsaturated fats. These oxo-biodegradable additives are incorporated into materials formed from the polymers by addition of the additive to the molten polymer during extrusion thereof, followed by molding of the thermoplastics containing the additive. The oxo-biodegradable additives then function to accelerate the breakdown of the polymers into molecules that could be consumed by biodegradative living organisms.

However, as the oxo-biodegradable additive is incorporated into the extrudate, a certain amount of additive is required to distribute the additive throughout the material and thus render the material oxo-biodegradable. Indeed, some requirements for oxo-biodegradable material do not require sufficient quantities of a particular product to justify the fixed cost of extruding or molding a custom part using resin blended with oxo-biodegradable additives.

Another drawback to the current technology is that the use of high extrusion temperatures limits the types of additives that can be used. Also, certain properties required in a film, sheet, or molded object may not be attainable with the oxo-biodegradable additives currently available. Further, there are certain materials that cannot be formed by extrusion methods, and thus these materials cannot be rendered oxo-biodegradable by the current technology that requires incorporation of oxo-biodegradable additives therein during the extrusion process.

Therefore, there is a need in the art for new and improved ways of rendering a material oxo-biodegradable. It is to such methods of causing, enhancing, and/or expediting oxo-biodegradation, as well as to treated materials for which oxo-biodegradation has been caused, enhanced, and/or expedited, that the presently disclosed and claimed inventive concept(s) is directed.

### DETAILED DESCRIPTION

Before explaining at least one embodiment of the presently disclosed and claimed inventive concept(s) in detail, it is to be understood that the presently disclosed and claimed inventive concept(s) is not limited in its application to the details of construction and the arrangement of the components or steps or methodologies set forth in the following description or illustrated in the drawings. The presently disclosed and claimed inventive concept(s) is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed and claimed herein can be made and executed without undue experimentation in light of the present disclosure. While the articles and methods of the presently disclosed and claimed inventive concept(s) have been described in terms of preferred embodiments, it will be apparent to those of skill in the art that variations may be applied to the articles and/or methods and in the steps or in the sequence of steps of the method described herein without departing from the concept, spirit, and scope of the presently disclosed and claimed inventive concept(s). All such similar substitutes and modifications apparent to those skilled in the art are deemed to be within the spirit, scope, and concept of the presently disclosed and claimed inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one”, but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. For example, but not by way of limitation, when the term “about” is utilized, the designated value may vary by plus or minus twelve percent, or eleven percent, or ten percent, or nine percent, or eight percent, or seven percent, or six percent, or five percent, or four percent, or three percent, or two percent, or one percent. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

The term “oxo-biodegradation” as used herein will be understood to refer to a two-step process that combines a degradative process step with a biodegradative process step in the environment. Oxo-biodegradation includes a first, abiotic process that is combined with a second, biotic process step. The first step (i.e., the abiotic process step) is a chemical process that requires the presence of oxygen so that degradation of a material can occur through oxidation. The second step (i.e., the biotic process step) is a biological process that requires the presence of at least one biodegradative living organism that is capable of biodegradative consumption of the degraded material produced in the first step of the process. Once the degradative step of oxo-biodegradation is initiated, the degradative and biodegradative processes can occur simultaneously and/or wholly or partially sequentially. Oxo-biodegradation requires the presence of a prodegradant (i.e., an “agent of oxo-biodegradation,” as described in detail herein below) that speeds up the molecular breakdown of a material during the first step of oxo-biodegradation and that incorporates oxygen atoms into the resulting low molecular mass. This chemical change enables the further breakdown of the material in the second step by biodegradative living organism(s). In this manner, for example, but not by way of limitation, the degradation process (i.e., the first step of oxo-biodegradation) is shortened from a period of hundreds of years to a period of years or even months; thereafter, the rate of the biodegradative process (i.e., the second step of oxo-biodegradation) depends on the biodegradative living organism(s) present in the environment.

The terms “agent of oxo-biodegradation,” “oxo-biodegradative additive,” and “prodegradant” may be used interchangeably herein and will be understood to refer to any molecule that can cause, enhance, and/or expedite at least the first step (i.e., the abiotic, degradative process) of oxo-biodegradation. For example, but not by way of limitation, the agent of oxo-biodegradation may be an oxidizable molecule or a molecule capable of receiving electrons. In addition to causing, enhancing, and/or expediting the first step of oxo-biodegradation, the molecule may also enhance and/or expedite the second step (i.e., biodegradative process) of oxo-biodegradation.

The term “living organism” as used herein will be understood to include any organism capable of reproduction. Certain embodiments of the presently disclosed and claimed inventive concept(s) may include the use of one or two types of living organisms. A first type of living organism that may be utilized herein is a living organism that is capable of consuming article(s) disclosed herein by biodegradative means (referred to herein as a “biodegradative living organism”). A second type of living organism that may be utilized herein is a living organism that functions to stimulate and/or attract the first type of living organism (i.e., biodegradative organism) to article(s) as disclosed herein and/or enhance and/or expedite the rate of consumption of the article(s) by the biodegradative living organism; these secondary living organisms may also be biodegradative, or they may be non-biodegradative. Non-limiting examples of living organisms (both biodegradative and non-biodegradative) contemplated for use in accordance with the presently disclosed and claimed inventive concept(s) include microbes (including but not limited to, bacteria, viruses, protozoa, algae, and fungi), worms (such as but not limited to, earthworms), termites, ants, and the like. Living organisms functioning as described herein above are well known in the art, and it is well within the skill of a person of ordinary skill in the art to identify and select organisms capable of functioning as the first and second types of living organisms in accordance with the presently disclosed and claimed inventive concept(s). Thus, no further discussion on the identification and selection of the first and second types of living organisms is deemed necessary.

The terms “microbe” and “microorganism” may be utilized interchangeably herein. Selection of microorganisms capable of functioning as first and/or second types of living microorganisms in accordance with the presently disclosed and claimed inventive concept(s) is well within the ability of a person having ordinary skill in the art. Thus, no further discussion on the identification and selection of first and/or second types of living microorganisms is deemed necessary.

The term “type” in the phrase “type of living organism” as used herein will be understood to refer to a function of the living organism rather than a structural characteristic of the living organism. That is, the term “first type of living organism” will be understood to refer to the biodegradative ability of that living organism, while the term “second type of living organism” will be understood to refer to the ability of that living organism to stimulate and/or attract the biodegradative organism and/or enhance and/or expedite the rate of consumption of the biodegradative organism. The term “type” in the phrase “type of living organism” is not to be construed as referring to a single species; indeed, a combination of multiple species that share a similar function may be encompassed within the scope of a single “type of living organism,” as the term is utilized herein.

The combination of the terms “nutrients,” “attractants,” “stimulants,” and “hormones” encompass any substance for which a biodegradative living organism utilized herein has an affinity. For example, but not by way of limitation, these substances may provide nutrition to the organism, attract the organism, stimulate the organism's metabolism, stimulate the organism's consumption rate, and/or stimulate the organism's function in any other manner that would be conducive to the consumption of an article by the biodegradative living organism.

The term “associate” as used herein will be understood to refer to the direct or indirect connection of two or more items.

Turning now to the presently disclosed and claimed inventive concept(s), one embodiment thereof is directed to an assembly that includes an article formed of a material and a composition disposed on at least a portion of the material. The composition includes at least one agent of oxo-biodegradation, whereby the composition causes, enhances, and/or expedites oxo-biodegradation of at least a portion of the treated material. The agent of oxo-biodegradation may cause, enhance, and/or expedite oxo-biodegradation of the composition in which the agent is disposed, the material to which the composition is applied, or both the composition in which the agent is disposed and the material to which the composition is applied. In addition, the agent of oxo-biodegradation may also cause, enhance, and/or expedite oxo-biodegradation of one or more other substance(s)/agent(s) that may be present in the composition (i.e., a carrier or other substance(s)/agent(s) described in further detail herein below).

Any article for which oxo-biodegradation of at least a portion thereof is desired may be utilized in accordance with the presently disclosed and claimed inventive concept(s). In particular, the degradation/consumption of disposable articles is typically desired and thus encompassed within the scope of the presently disclosed and claimed inventive concept(s). Non-limiting examples of articles for which at least a portion thereof may be rendered oxo-biodegradable and thus may be utilized in accordance with presently disclosed and claimed inventive concept(s) include, but are not limited to, a film, a sheet, a floral packaging material, a floral container, a floral foam, a growing pot, a flower pot, a growing tray, a plug tray, a plug holder, a vase, a decorative grass, a packaging material, a food service product, a cup, a bag, a sleeve, a food wrapper, a food container, a liquid container, a food utensil, a carrying tray, a plate, a bowl, a tablecloth, a place mat, toilet paper, a paper towel, clothing, a non-woven cloth or cloth-like product, pharmaceutical packaging, a medical device, a filter, a cartridge case (in whole or in part), cartridge and firearm wad or wadding, a ground cover film (including woven and non-woven films), a shrink wrap, a stretch wrap, combinations thereof, and the like.

Particular examples of floral packaging material include, but are not limited to, a sheet of wrapping material, a roll of wrapping material, a bag, a sleeve (such as but not limited to plant sleeves, cut flower sleeves, dried materials sleeves, artificial floral materials sleeves, bouquet sleeves, herb sleeves, floral grouping sleeves, pot covering sleeves, as well as produce sleeves), a wrapper for a floral grouping or a flower pot, a preformed flower pot cover, combinations thereof, and the like.

Particular examples of food service products include, but are not limited to, plastic and plastic coated plates; drinking cups and glasses made from plastics and/or plastic coated materials; plastic food utensils such as plastic forks, knives, and spoons; table cloths (wherein at least a portion thereof is formed of plastic), place mats (wherein at least a portion thereof is formed of plastic), and the like. Particular examples of liquid containers include, but are not limited to, plastic milk or juice containers, coated milk or juice containers, milk or juice cartons, beverage containers, beverage pouches, and the like.

Particular examples of non-woven cloth and cloth-like products include, but are not limited to, bed sheets, surgical gowns, patient gowns, pillow cases, aprons, and the like. Particular examples of pharmaceutical packaging include, but are not limited to, blister pack film and sheets, bags and trays containing medical devices and/or pharmaceuticals, and the like. Particular examples of medical devices include, but are not limited to, syringes, needles (including portion(s) thereof), catheters, IV fluid bags, IV tubing, gloves, and the like.

Particular examples of packaging materials include, but are not limited to, cosmetics, pharmaceutical, and food supplement packaging, decorative packaging, and the like. Particular examples of filters include, but are not limited to, air, water, fuel, oil, water purification, and air conditioning filters, and the like. Particular examples of wrappers include, but are not limited to, twist wraps, pouches, form fill packages, and the like. Particular examples of bags include ZIPLOC® bag-type products (SC Johnson and Son, Inc., Racine, Wis.), bag zippers, snack bags (including laminated and unlaminated bags), cooking bags, and the like.

The article may be formed of any material wherein at least a portion thereof is capable of being rendered oxo-biodegradable upon application of at least an agent of oxo-biodegradation thereto. The treated portion of the material from which the article is formed may be a substantially non-biodegradable material that is rendered both biodegradable and oxo-biodegradable by application of the composition containing at least the agent of oxo-biodegradation thereto. Alternatively, the treated portion of the material from which the article is formed may be a material that is substantially biodegradable but substantially non-oxo-biodegradable; in this instance, the treated material is rendered oxo-biodegradable by application of the composition containing at least the agent of oxo-biodegradation thereto. In certain embodiments, only the portion of the material that is treated may be substantially degraded by oxo-biodegradation, while the remaining portion of the material is not substantially degraded by oxo-biodegradation. Alternatively, application of the composition containing at least the agent of oxo-biodegradation to the material may also cause, enhance, and/or expedite oxo-biodegradation of at least a portion of the untreated material. The oxo-biodegradation of the untreated material may be the result of the agent of oxo-biodegradation penetrating into at least a portion of the untreated material.

Examples of materials that may be utilized to form the articles of the presently disclosed and claimed inventive concept(s) include, but are not limited to, a polyolefin, paper, a natural fiber, a degradable polymer, a biodegradable polymer, and combinations and laminates thereof. Particular examples of polyolefins include, but are not limited to, polypropylene, polyethylene, polystyrene, polyvinyl acetate, polyvinyl chloride, combinations thereof, and the like. Particular examples of biodegradable polymers include, but are not limited to, polyvinyl alcohol, polyesters, polycaprolactone, polyhydroxyalkanoates, polylactic acid, polyamides, combinations thereof, and the like.

The agent of oxo-biodegradation may be any molecule that can cause, enhance, and/or expedite at least the first step (i.e., the abiotic, degradative process step) of oxo-biodegradation. For example, but not by way of limitation, the agent of oxo-biodegradation may be an oxidizable molecule or a molecule capable of receiving electrons (i.e., an electron recipient). The agent may catalyze both stages of the redox cycle of degradation (i.e., the oxidation and reduction stages). In addition to causing, enhancing, and/or expediting the first step of oxo-biodegradation, the molecule may also enhance and/or expedite the second step (i.e., the biodegradative process step) of oxo-biodegradation. Non-limiting examples of agents of oxo-biodegradation include transition metal salts, transition metal salts of fatty acids, transition metal chelates, unsaturated fatty acids, unsaturated fats, oxidative enzymes, combinations thereof, and the like. When a metal is present in the agent of oxo-biodegradation, the metal must have a plural ionic character (i.e., a transition metal). The rate of the first step of oxo-biodegradation can be controlled by the addition of multiple agents of oxo-biodegradation and/or multiple types of agents of oxo-biodegradation, whereby the presence of multiple agents may expedite the rate of degradation.

Non-limiting examples of transition metal salts that may be utilized as an agent of oxo-biodegradation in accordance with the presently disclosed and claimed inventive concept(s) include transition metal stearates, such as but not limited to, chromium stearate, cobalt stearate, copper stearate, iron stearate, manganese stearate, nickel stearate, titanium stearate, vanadium stearate, combinations thereof, and the like; transition metal fumarates, such as but not limited to, chromium fumarate, cobalt fumarate, copper fumarate, iron fumarate, manganese fumarate, nickel fumarate, titanium fumarate, vanadium fumarate, combinations thereof, and the like; transition metal maleates, such as but not limited to, chromium maleate, cobalt maleate, copper maleate, iron maleate, manganese maleate, nickel maleate, titanium maleate, vanadium maleate, combinations thereof, and the like; and transition metal citrates, such as but not limited to, chromium citrate, cobalt citrate, copper citrate, iron citrate, manganese citrate, nickel citrate, titanium citrate, vanadium citrate, combinations thereof, and the like.

Non-limiting examples of transition metal salts of fatty acids that may be utilized as an agent of oxo-biodegradation in accordance with the presently disclosed and claimed inventive concept(s) include transition metal salts of saturated fatty acids, such as but not limited to, transition metal salts of caprylic acid (octanoic acid), capric acid (decanoic acid), lauric acid (dodecanoic acid), myristic acid (tetradecanoic acid), palmitic acid (hexadecanoic acid), stearic acid (octadecanoic acid), arachidic acid (eicosanoic acid), behenic acid (docosanoic acid), lignoceric acid (tetracosanoic acid), cerotic acid (hexacosanoic acid), combinations thereof, and the like; and transition metal salts of unsaturated fatty acids, such as but not limited to, transition metal salts of myristoleic acid, palmitoleic acid, sapienic acid, oleic acid, elaidic acid, vaccenic acid, linoleic acid, linoelaidic acid, arachidonic acid, eicosapentaenoic acid, erucic acid, docosahexaenoic acid, combinations thereof, and the like.

Non-limiting examples of chelating agent that may be utilized with transition metals as an agent of oxo-biodegradation in accordance with the presently disclosed and claimed inventive concept(s) include 2,2′-Bipyridine, 1,10-Phenanthroline, an (aminomethyl)pyridine, a polyfunctional amine, a chelating agent present in an alkyd, combinations thereof, and the like.

Non-limiting examples of unsaturated fats/fatty acids that may be utilized as an agent of oxo-biodegradation in accordance with the presently disclosed and claimed inventive concept(s) include mono-unsaturated fats/fatty acids, such as but not limited to, myristoleic acid, palmitoleic acid, sapienic acid, oleic acid, elaidic acid, vaccenic acid, erucic acid, combinations thereof, and the like; and poly-unsaturated fats/fatty acids, such as but not limited to, linoleic acid, linoelaidic acid, arachidonic acid, eicosapentaenoic acid, docosahexaenoic acid, combinations thereof, and the like.

Non-limiting examples of oxidative enzymes that may be utilized in accordance with the presently disclosed and claimed inventive concept(s) include lignin peroxidases, cytochrome oxidases, manganese peroxidases, laccases, combinations thereof, and the like.

Another non-limiting example of an agent of oxo-biodegradation comprises an enzyme that catalyzes the hydrolysis of a biodegradable polymer. Particular examples include, but are not limited to, esterases, hydrolases, combinations thereof, and the like.

The composition may be provided in any form so long as the composition may be disposed on at least a portion of at least one surface of a material and can function in accordance with the presently disclosed and claimed inventive concept(s). In certain embodiments, the composition may be a coating that includes the at least one agent of oxo-biodegradation, and may further include one or more additional substances/agents, as described in further detail herein below. Examples of composition/coating forms that may be utilized in accordance with the presently disclosed and claimed inventive concept(s) include, but are not limited to, a powder, a liquid, a solid, a gas, a hot melt, an extruded layer, an ink, a lacquer, an adhesive, a powder coating, an electronically and/or electrostatically applied, adhered, and/or retained coating, a heat sealing lacquer, a pressure sealing lacquer, a tape, a cold seal adhesive, a plasma, any combination of two or more of the above forms, and the like. In particular, non-limiting examples, the composition/coating may be a powder that is comprised of particles of one or more solids, or the composition/coating may be a liquid that upon or subsequent to application becomes a solid and adheres to a portion of the material to which it is applied.

The composition containing the agent of oxo-biodegradation may be disposed on at least a portion of at least one surface of the material from which the article is formed. Upon disposal, the composition may remain on and/or near the surface of the material; alternatively (and/or in addition thereto), the composition may be absorbed and/or adsorbed by the material. The composition may further penetrate beyond at least a portion of the surface of the material and into at least a portion of the material. For example, but not by way of limitation, the composition may also be capable of penetrating beyond at least a portion of the surface of the material and thus render a portion of the untreated material oxo-biodegradable as well.

When the article is formed of a laminate, the composition may be applied to at least a portion of at least one surface of the laminate and/or at least a portion of at least one surface of one or both of the two or more layers that form the laminate. In one particular, non-limiting embodiment, the composition containing the agent of oxo-biodegradation may further comprise an adhesive that connects at least two layers of the laminate to one another. When the agent of oxo-biodegradation is not present on a surface of a material but rather is solely disposed in between two layers of material, exposure to sources of oxygen and water will be required to activate the agent of oxo-biodegradation in the composition. In one embodiment, the composition may include at least one source of oxygen and/or at least one source of water (as described in further detail herein after) that are also disposed between the two layers. Alternatively, a method of use of this material containing the composition disposed between the two layers may include the step of exposing the composition disposed between the two layers to air at a certain point during use of the article so that the exposure to air can activate the agent of oxo-biodegradation. In a particular, non-limiting example of this embodiment, the article may include a removable portion (such as, but not limited to, a tab) and/or an opening portion (such as, but not limited to, an opening strip) that can be pulled to expose the composition disposed in between the two layers of material to air.

The composition may be disposed on at least a portion of the material in any desired amount and any desired shape and/or pattern, so long as the agent of oxo-biodegradation present therein is capable of functioning in accordance with the presently disclosed and claimed inventive concept(s) (i.e., is capable of causing, enhancing, and/or expediting oxo-biodegradation of at least the treated material). In certain embodiments, the composition is disposed on the material in a seemingly random arrangement; alternatively, the composition is disposed on the material in a controlled manner that provides a desired surface area density sufficient to allow for oxo-biodegradation of at least the treated portion of the article/material. In addition, the composition may be disposed on the article/material in any desired coating, shape, and/or pattern, including but not limited to, dots and strips as well as fanciful shapes and/or patterns.

In certain embodiments, the composition containing the at least one agent of oxo-biodegradation may further include a carrier in which the at least one agent of oxo-biodegradation is disposed. Non-limiting examples of carriers that may be utilized in accordance with the presently disclosed and claimed inventive concept(s) include an acrylic lacquer, a nitrocellulose lacquer, an epoxy coating, a natural rubber-containing coating, a polyolefin coating, a synthetic rubber coating, a latex coating, a soponified substance, a soap, a fat, an oil, a wax, like carriers, combinations thereof, and the like. Other non-limiting examples of carriers include an extrusion coating, a coextruded layer, a hot melt, combinations thereof, and the like; these latter carriers differ from the prior art (in which a prodegradant is added to polyolefins during extrusion to promote oxo-biodegradation of the polymer structure) in that these carriers promote oxo-biodegradation of an adjacent material on which the composition containing the at least one agent of oxo-biodegradation and the carrier is disposed. Another non-limiting example of a carrier is a laminated web or other structure to which the material for which oxo-biodegradation is desired is attached. In one embodiment, the laminated web or other structure may have the agent of oxo-biodegradation disposed on at least one surface thereof; in an alternative embodiment, the laminated web or other structure may have the agent of oxo-biodegradation incorporated therein (such as incorporated into a melted polymer structure). Like the other carriers mentioned above, the laminated web-type carrier differs from the prior art (in which a prodegradant is added to polyolefins during extrusion to promote oxo-biodegradation of the polymer structure) in that the laminated web-type carriers promote oxo-biodegradation of an adjacent material that is laminated or otherwise attached to the carrier.

In addition to the agent of oxo-biodegradation, one or more additional agents/substances may be utilized in accordance with the presently disclosed and claimed inventive concept(s). These agents/substances may be included in the composition in which the agent of oxo-biodegradation is disposed; alternatively, these agents/substances may be applied separately to the material or may be incorporated/impregnated within the material. These agents/substances may function to enhance the rate of oxo-biodegradation by enhancing either or both steps of the oxo-biodegradative process. Examples of these types of agents/substances include, but are not limited to, at least one source of oxygen; at least one source of water; at least one agent of photodegradation; at least one substance that activates the agent of oxo-biodegradation; a substance that attracts and/or nourishes a biodegradative living organism and/or enhances and/or expedites a rate of consumption by the biodegradative living organism; at least one biodegradative living organism; at least one living organism that stimulates and/or attracts a biodegradative living organism and/or enhances and/or expedites a rate of consumption by the biodegradative living organism; combinations thereof, and the like.

For oxo-biodegradation to occur, an oxygen source and a water source must be present. Therefore, one or more of these sources may be included in the composition in which the agent of oxo-biodegradation is disposed, or one or more of these sources may be applied separately to the material or may be incorporated/impregnated within the material. Particular examples of oxygen sources include, but are not limited to, air bubbles, oxygen bubbles, ozonides, peroxides, hydrogen peroxide, emulsified air, emulsified oxygen, emulsified ozone, encapsulated air, encapsulated oxygen, combinations thereof, and the like. Particular examples of water sources include, but are not limited to, liquid water and/or water vapor containing bubbles, water droplets, emulsified water, emulsified water vapor, encapsulated water, encapsulated water vapor, combinations thereof, and the like. Particular examples of agents of photodegradation include, but are not limited to, natural sunlight, an ultraviolet light source, combinations thereof, and the like. Any of the emulsified substances may be emulsified alone or with other substance(s) (i.e., water, water vapor, oxygen, and/or air may be emulsified with a coating and/or the agent of oxo-biodegradation).

It may also be desirable to dispose the agent of oxo-biodegradation in the composition in an inactive form, so that oxo-biodegradation of the article does not begin immediately upon contact with the agent of oxo-biodegradation and the oxygen and water sources. In this manner, a substance that activates the agent of oxo-biodegradation may be included in the composition in which the agent of oxo-biodegradation is disposed, or applied separately to the material or may be incorporated/impregnated within the material. In this manner, the initiation of oxo-biodegradation can be controlled. Examples of substances that can activate an agent of oxo-biodegradation include, but are not limited to, a source of water, a source of oxygen, a promoter of an agent of oxo-biodegradation (as described in detail herein below), combinations thereof, and the like. Optionally, the agent of oxo-biodegradation may be encapsulated so as to render the agent inactive until released therefrom.

In a like manner, promoters and/or inhibitors (and/or competitors) of the agent of oxo-biodegradation may also be present to allow for control of the rate and/or onset of oxo-biodegradation of the article. Promoters of agents of oxo-biodegradation may affect either or both stages of the redox cycle of reduction. Non-limiting examples of promoters of agents of oxo-biodegradation include other agents of oxo-biodegradation (as described in detail herein above) and electron donors, such as but not limited to, an electrical corona discharge treatment, a plasma discharge treatment, combinations thereof, and the like.

An inhibitor/competitor of an agent of oxo-biodegradation may be any substance/agent that can bind or otherwise affect an agent of oxo-biodegradation so that the activity thereof is substantially diminished, or so that the agent is no longer catalytically active (i.e., a proton donor or an electron scavenger). Non-limiting examples of inhibitors/competitors of agents of oxo-biodegradation include acids; hydrogen and other free radicals; sulfides, such as but not limited to, hydrogen sulfide; antioxidants, such as but not limited to, IRGANOX® (BASF, Florham Park, N.J.) and polyphenols; combinations thereof; and the like.

As stated herein above, a substance that functions to attract and/or nourish the biodegradative living organism(s) and/or enhance and/or expedite the rate of consumption of the treated material by the biodegradative living organism(s) may also be utilized in accordance with the presently disclosed and claimed inventive concept(s). The substance may be a nutrient, a stimulant, an attractant, a hormone, another organism (i.e., living or non-living, biodegradative or non-biodegradative), and/or any combinations thereof. The substance may enhance and/or expedite consumption of the treated material by biodegradative living organism(s) by any method known in the art or otherwise contemplated herein. Non-limiting examples of enhancing/expediting methods include: (a) attracting biodegradative living organism(s) to the article; (b) increasing the speed of consumption of the article by biodegradative living organism(s); (c) increasing activity of biodegradative living organism(s); and (d) enhancing the health of biodegradative living organism(s). Particular, non-limiting examples of substances that function in this manner include a metal, a metal salt, a metal stearate, theobromine, theophylline, mildronate, 3-(2,2,2-trimethylhydraziniumyl) propionate, caffeine, starch, sugar, tea, tea leaves, chocolate, cocoa, coffee, coffee grounds (i.e., coffee grounds used to make a coffee beverage), ground coffee beans, a short chain saturated fat, a grain, a thyroid hormone, a pheromone, dichloroacetic acid (DCA), sodium dichloroacetate, an enzyme, combinations thereof, and the like.

When present, the first type of living organism (i.e., biodegradative living organism) functions to cause, enhance, and/or expedite the second step of the oxo-biodegradative process. When present, the second type of living organism functions to stimulate and/or attract biodegradative living organism(s) and/or enhance and/or expedite a rate of consumption by biodegradative living organism(s). The second type of living organism may also be biodegradative, or the second type of living organism may be non-biodegradative. Alternatively, the term “second type of living organism” may encompass a combination of biodegradative and non-biodegradative living organisms.

The first and second types of living organisms utilized in accordance with the presently disclosed and claimed inventive concept(s) (whether biodegradative or non-biodegradative) may be utilized at any point during their life cycle. For example, but not by way of limitation, eggs, embryos, youth, and/or adult organisms may be utilized. In addition, the living organisms (regardless of life stage) may be disposed in a package, covering, or the like. The package/covering may be naturally-occurring, such as but not limited to, a cocoon or spore (a non-limiting example of which is an earthworm cocoon). Optionally, a package/covering may be added to the living organism; for example, but not by way of limitation, a living organism may be encapsulated. In addition, it may be desirable to encapsulate a living organism that is already disposed within a naturally-occurring package/covering.

A non-limiting example of biodegradative living organisms contemplated for use herein are earthworms, such as those typically present in gardens and flower beds, including but not limited to the species Lumbricus terrestris, Eisenia fetida, and/or Eisenia hortensis. Another non-limiting example of earthworms that may be utilized in accordance with the presently disclosed and claimed inventive concept(s) include the VERMIPOD™ encapsulated earthworm cocoons (VermiPod, LLC, Highland, Ill.). Production of encapsulated earthworm cocoons is described in detail in U.S. Pat. No. 6,834,614, issued to Kreitzer on Dec. 28, 2004, the entire contents of which are expressly incorporated herein by reference.

Other examples of living organisms possessing biodegradative abilities are well known in the art. Based on the disclosure provided herein, it is well within the skill of a person of ordinary skill in the art to select one or more particular types of biodegradative living organisms for use within the scope of the presently disclosed and claimed inventive concept(s), and thus no further description thereof is deemed necessary.

The use of combinations of biodegradative living organisms (whether as the first type of living organism and/or as the first and second types of living organisms) also falls within the scope of the presently disclosed and claimed inventive concept(s). One such combination includes multiple species of living organisms. A non-limiting example of such a combination includes earthworms with at least one microbe (such as bacteria); however, any combination of species of living organisms described or otherwise contemplated herein may be utilized (including two or more species, three or more species, four or more species, five or more species, as well as higher combinations of species). The combination may be formed of the first and second types of living organisms, or a single “type” of living organism may include a combination of multiple species.

In addition, combinations of living organisms (whether first and/or second types of organisms) at different stages in their life cycles may be utilized. Also, combinations of living organisms in which only a portion thereof contains a naturally-occurring packaging/covering may be utilized herein, as well as combinations of encapsulated and non-encapsulated living organisms. Further, any combination of the above combinations (such as but not limited to, multiple species of living organisms, each present at more than one stage in their life cycle and in both encapsulated and non-encapsulated forms) is encompassed by the scope of the presently disclosed and claimed inventive concept(s).

It may be desirable to provide biodegradative living organism(s) in a “biodegradably inactive form,” so that consumption of the article does not begin immediately upon contact with the biodegradative living organism(s). Rather, it may be desired that the biodegradative living organism(s) remain in a somewhat dormant state until “activated” by contact with a particular agent (such as but not limited to, water and/or soil). Once the biodegradative living organism(s) is brought into contact with the particular agent, the biodegradative living organism(s) become biodegradably active and begin consumption of the article. In a non-limiting embodiment, the biodegradative living organism(s) may be rendered biodegradably inactive by the package/covering disposed thereabout (such as but not limited to, a capsule or cocoon).

Thus, the use of combinations of biodegradative and non-biodegradative living organisms (whether as the second type of living organism and/or as the first and second types of living organisms) also falls within the scope of the presently disclosed and claimed inventive concept(s). A non-limiting example of such a combination includes biodegradative earthworms with at least one non-biodegradative microbe (such as but not limited to, bacteria, viruses, protozoa, algae, or fungi); however, any combination of species of biodegradative and non-biodegradative living organisms described or otherwise contemplated herein may be utilized (including two or more species, three or more species, four or more species, five or more species, as well as higher combinations of species). In addition, combinations of biodegradative and non-biodegradative living organisms at different stages in their life cycles may be utilized. Also, combinations of biodegradative and non-biodegradative living organisms in which only a portion thereof contain a naturally-occurring packaging/covering may be utilized herein, as well as combinations of encapsulated and non-encapsulated living organisms. Further, any combination of the above combinations (such as but not limited to, multiple species of living organisms, each present at more than one stage in their life cycle and in both encapsulated and non-encapsulated forms) is encompassed by the scope of the presently disclosed and claimed inventive concept(s).

In certain embodiments, any of the compositions containing an agent of oxo-biodegradation may be water proof and/or water resistant, or the compositions may be applied to a water proof and/or water resistant material. Thus, certain embodiments of the presently disclosed and claimed inventive concept(s) are directed to materials that are made water proof and/or water resistant by applying a water proof and/or water resistant composition containing an agent of oxo-biodegradation thereto, and/or by laminating the material to a second material that is water proof and/or water resistant and that has an agent of oxo-biodegradation disposed thereon and/or incorporated therein. For example, but not by way of limitation, paper and/or cardboard may have such a composition disposed thereon and/or be laminated to such a material, thereby rendering the resultant product both oxo-biodegradable and water proof and/or water resistant.

Certain embodiments of the presently disclosed and claimed inventive concept(s) are directed to a kit. The kit may contain any of the articles described herein above or otherwise contemplated herein as well as any of the compositions comprising the at least one agent of oxo-biodegradation as described herein above or otherwise contemplated herein. The composition may be disposed upon the article/material, or the composition may be supplied as a separate component of the kit. In addition, any of the other substances/agents mentioned herein above (i.e., oxygen/water source(s), agent(s) of photodegradation, biodegradative and/or non-biodegradative living organism(s), substance(s) capable of activating the agent of oxo-biodegradation, and/or substance(s)/agent(s) that attracts/nourishes organism and/or enhances/expedites rate of consumption thereby) may also be present in the kit. When present, one or more of these additional substances/agents may be provided as one or more separate components of the kit; alternatively (or in combination therewith), one or more of these additional substances/agents may be disposed in the composition containing the agent of oxo-biodegradation and/or the substance(s)/agent(s) may be disposed on and/or incorporated within the article/material.

In certain embodiments, the kits of the presently disclosed and claimed inventive concept(s) may include instructions for obtaining the first and/or second type of living organism. In addition, the kit may further include one or more of various instructions related to activating at least a portion of the oxo-biodegradative process; that is, the kit may include instructions for activating the agent of oxo-biodegradation to initiate the first step of the oxo-biodegradative process, instructions for activating the biodegradative living organism to begin the second step of the oxo-biodegradative process, and/or instructions for causing the second type of living organism to begin stimulating and/or attracting the first type of living organism and/or enhancing and/or expediting the rate of consumption of the first type of living organism.

Any type of written wording and/or pictorial drawings capable of conveying a method of obtaining the living organism(s) and/or activating at least a portion of the oxo-biodegradative process may be utilized as the instructions described and claimed herein. Non-limiting examples of instructions include a hard copy of pictorial depictions and/or written detailed instructions; a website and/or e-mail address for obtaining detailed instructions and/or for placing an online order; a post card for requesting the organism(s) through the mail; a bar code (such as, but not limited to, a QR code) that is readable by an imaging device/code reader; instructions for gathering naturally-occurring organism(s) from their natural habitat; and any combination thereof.

In addition, the kit may include, for example but not by way of limitation, one or more materials in which the article may be disposed to provide an environment conducive to the article's oxo-biodegradation. In this manner, the composition may be applied to the article at a time when consumption thereof is desired.

In certain embodiments, separate and/or unattached elements of the kit may be associated with one another via a packaging. The term “packaging” as used herein will be understood to include any element(s) that associate the content(s) of the kits contemplated herein with one another. Non-limiting examples of packaging that may be utilized in accordance with the presently disclosed and claimed inventive concept(s) include a bag, a wrapper, adhesive, cohesive, tape, a flexible container, a rigid container, stretch wrap, shrink wrap, combinations thereof, and the like.

Another embodiment of the presently disclosed and claimed inventive concept(s) is directed to a method of causing, enhancing, and/or expediting biodegradation of an article. The method includes disposing any of the compositions containing an agent of oxo-biodegradation, as described herein above or otherwise contemplated herein, on at least a portion of any of the materials/articles described herein above or otherwise contemplated herein. In certain embodiments, the composition and/or material/article may further comprise any of the additional agents/substances described herein above and/or otherwise contemplated herein. In another embodiment, the method may further comprise the step of disposing at least one of the additional agents/substances on at least a portion of the material/article.

The method may further include the step of placing the article in an environment conducive to oxo-biodegradation thereof. In addition, the article/material may be directly subjected to consumption in its originally provided form, or the article/material may be divided into smaller pieces/segments (such as but not limited to, by cutting or tearing) for ease and/or expedition of oxo-biodegradation thereof.

Also, the method may further include the step of activating the agent of oxo-biodegradation, activating one of the other substances/agents, and/or activating one of the living organism(s).

Given that agents of oxo-biodegradation and the various living organisms capable of use herein are known in the art, it is well within the skill level of a person of ordinary skill in the art to determine the ideal oxo-biodegradation conditions to utilize in the methods disclosed and claimed herein. For example, but not by way of limitation, instructions for determining the ideal consumption/biodegradation conditions to utilize with the VERMIPOD™ encapsulated earthworm cocoons (VermiPod, LLC., Highland, Ill.) are described in detail in U.S. Pat. No. 7,478,604, issued to Kreitzer on Jan. 20, 2009, the entire contents of which are expressly incorporated herein by reference. The ideal consumption/biodegradation conditions for other biodegradative organisms that can be utilized in accordance with the presently disclosed and claimed inventive concept(s) are well known in the art and/or can easily be determined by a person having ordinary skill in the art, given the description provided herein. Therefore, no further description of determining these conditions is deemed necessary.

Another embodiment of the presently disclosed and claimed inventive concept(s) is directed to a container such as but not limited to, a floral foam, a growing pot, a flower pot, a growing tray, a plug tray, a plug holder, a vase, combinations thereof, and the like. At least a portion of the container is produced from an oxo-biodegradable polymer material. The polymer material may be made oxo-biodegradable by extrusion of at least one agent of oxo-biodegradation with the polymer material, and thereby providing a polymer material having the at least one agent of oxo-biodegradation incorporated/impregnated therewithin. Alternatively, the polymer material may be made oxo-biodegradable by any of the methods described herein above or otherwise contemplated herein.

The polymer material may be any of the materials described herein above or otherwise contemplated herein. In addition, when the polymer material is a laminated material, at least a portion of at least one layer of the laminated material is an oxo-biodegradable polymer material. The at least one layer may be made oxo-biodegradable by extrusion of at least one agent of oxo-biodegradation with the polymer material, or the at least one layer may be made oxo-biodegradable by any of the methods described herein above or otherwise contemplated herein.

Another embodiment of the presently disclosed and claimed inventive concept(s) is directed to a method of use of the above-referenced containers; in the method, at least one of soil, a plant, a floral grouping, and a propagule is disposed in any of the containers (at least a portion of which is oxo-biodegradable) described herein above or otherwise contemplated herein.

While examples of sequences of method steps are provided herein, it is to be understood that the method steps, as well as the order of the steps, may vary. Any combination of method steps and sequences thereof may be utilized so long as the kits and/or assemblies described herein are capable of functioning in accordance with the presently disclosed and claimed inventive concept(s).

Thus, in accordance with the presently disclosed and claimed inventive concept(s), there have been provided assemblies and kits, as well as methods of producing and using same, that fully satisfy the objectives and advantages set forth hereinabove. Although the presently disclosed and claimed inventive concept(s) has been described in conjunction with the specific drawings, experimentation, results, and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s). Changes may be made in the construction and the operation of the various components, elements, and assemblies described herein, as well as in the steps or the sequence of steps of the methods described herein, without departing from the spirit and scope of the presently disclosed and claimed inventive concept(s) as defined in the following claims.

What is claimed:

1. A method of causing, enhancing, and/or expediting biodegradation of an article, the method comprising the step of:
disposing a composition on at least a portion of a surface of a material from which an article is formed, wherein the composition comprises at least one agent of oxo-biodegradation;
forming the material into at least one article, wherein the at least one article is selected from the group consisting of a floral packaging material, a floral container, a floral foam, a growing pot, a flower pot, a growing tray, a plug tray, a plug holder, a vase, a decorative grass, a packaging material, a food service product, a cup, a bag, a sleeve, a food wrapper, a food container, a liquid container, a food utensil, a carrying tray, a plate, a bowl, a tablecloth, a place mat, a non-woven cloth or cloth-like product, pharmaceutical packaging, a medical device, a filter, a cartridge case, cartridge and firearm wad or wadding, a ground cover film, a shrink wrap, a stretch wrap, and combinations thereof; and
wherein the composition causes, enhances, and/or expedites oxo-biodegradation of at least a portion of the article.

2. The method of claim 1, wherein the material from which at least a portion of the article is formed is a substantially non-biodegradable material and/or a substantially non-oxo-biodegradable material.

3. The method of claim 2, wherein the composition is only disposed on a portion of the material, and the remaining portion of the material is not substantially degraded by oxo-biodegradation.

4. The method of claim 2, wherein the composition is only disposed on a portion of the material, and wherein the composition causes, enhances, and/or expedites oxo-biodegradation of at least a portion of the untreated material.

5. The method of claim 1, wherein the at least one agent of oxo-biodegradation comprises a transition metal salt and/or a transition metal chelate.

6. The method of claim 5, wherein the transition metal salt is a transition metal stearate selected from the group consisting of chromium stearate, cobalt stearate, copper stearate, iron stearate, manganese stearate, nickel stearate, titanium stearate, vanadium stearate, and combinations thereof.

7. The method of claim 1, wherein the at least one agent of oxo-biodegradation comprises a transition metal salt of a fatty acid.

8. The method of claim 1, wherein the at least one agent of oxo-biodegradation comprises an unsaturated fat and/or unsaturated fatty acid.

9. The method of claim 1, wherein the at least one agent of oxo-biodegradation comprises an oxidative enzyme and/or an enzyme that catalyzes the hydrolysis of a biodegradable polymer.

10. The method of claim 1, wherein at least a portion of the material from which the article is formed is selected from the group consisting of a polyolefin, paper, a natural fiber, a degradable polymer, a biodegradable polymer, and combinations and laminates thereof.

11. The method of claim 1, wherein the composition penetrates beyond at least a portion of a surface of the material.

12. The method of claim 1, wherein the composition further comprises a carrier in which the at least one agent of oxo-biodegradation is disposed.

13. The method of claim 12, wherein the carrier is selected from the group consisting of an acrylic lacquer, a nitrocellulose lacquer, an epoxy coating, a natural rubber-containing coating, a polyolefin coating, a synthetic rubber coating, a latex coating, a soponified substance, a soap, a fat, an oil, a wax, and combinations thereof.

14. The method of claim 12, wherein the carrier is selected from the group consisting of an extrusion coating, a coextruded layer, a hot melt, a laminated structure, and combinations thereof.

15. The method of claim 1, further comprising the step of disposing at least one additional agent on at least a portion of the material from which the at least one article is formed, wherein the at least one additional agent comprises at least one of:
(a) at least one source of oxygen;
(b) at least one source of water;
(c) at least one agent of photodegradation;
(d) at least one substance capable of activating an agent of oxo-biodegradation;
(e) at least one biodegradative living organism;
(f) at least one living organism that stimulates and/or attracts a biodegradative living organism and/or enhances and/or expedites a rate of consumption by the biodegradative living organism; and
(g) a substance that attracts and/or nourishes a biodegradative living organism and/or enhances and/or expedites a rate of consumption by the biodegradative living organism.

16. The method of claim 15, wherein the at least one additional agent is present in the composition comprising the at least one agent of oxo-biodegradation.

17. The method of claim 15, wherein the at least one additional agent is incorporated in the material.

18. The method of claim 1, wherein the at least one article is selected from the group consisting of a floral packaging material, a floral container, and combinations thereof.

19. The method of claim 1, wherein the at least one article is selected from the group consisting of a floral foam, a growing pot, a flower pot, a growing tray, a plug tray, a plug holder, a vase, and combinations thereof.

20. The method of claim 1, wherein the at least one article is selected from the group consisting of a decorative grass, a packaging material, and combinations thereof.
